(19) United States
(12) Patent Application Publication
(10) Pub. No.: US 2010/0104624 A1
(43) Pub. Date: Apr. 29, 2010

(54) COMBINATION THERAPY USING PHOSPHODIESTERASE INHIBITORS

(76) Inventors: Peter LANGECKER, Monte Sereno, CA (US); Bryan T. Oronsky, Los Altos Hills, CA (US)

Correspondence Address: Goodwin Procter LLP, Exchange Place, Boston, MA, 02109, US

(21) Appl. No.: 12/483,137

(22) Filed: Jun. 11, 2009

Related U.S. Application Data
(60) Provisional application No. 61/060,751, filed on Jun. 11, 2008.

(51) Int. Cl. A61K 9/127; A61K 31/506; A61K 31/496; A61K 31/4985; A61K 31/53; A61K 36/00; A61K 31/135; A61K 31/522; A61K 31/195; A61K 31/405; A61K 31/404; A61K 31/403; A61K 31/454; A61K 31/4196; A61K 31/421; A61P 7/00

(52) U.S. Cl. 424/450; 514/275; 514/252.16; 514/250; 514/243; 424/725; 514/653; 514/263.34; 514/567; 514/419; 514/414; 514/411; 514/323; 514/383; 514/376; 514/654; 514/659

(57) ABSTRACT

Described here are compositions and methods for treating side-effects of vasodilator therapy. The compositions may include both a vasodilator and a side-effect alleviating agent in a single dosage form. Alternatively, the vasodilator and side-effect alleviating agent may be formulated separately, each in its own dosage form. The compositions may be packaged as kits for use with various medical conditions.

### CROSS-REFERENCE TO RELATED APPLICATIONS

This application claims priority to U.S. Provisional Patent Application Ser. No. 61/060,751 filed on Jun. 11, 2008, which is hereby incorporated by reference in its entirety.

### FIELD

Described here are compositions and kits for treating side-effects of vasodilation. The compositions and kits include a vasodilator in combination with an active agent that alleviates a side-effect of the vasodilator. Specifically, compositions and kits including a phosphodiesterase inhibitor as the vasodilator are described. Methods for combined administration of the vasodilator and the side-effect alleviating agent to treat various medical conditions are also described.

### BACKGROUND

Vasodilators are medications that relax the smooth muscle in blood vessel walls, thereby increasing the luminal diameter of the blood vessels (vasodilation). This relaxation lowers systemic blood pressure and increases blood flow to the tissues or organs affected by the vasodilator. There are various classes of vasodilators, one well known class being phosphodiesterase-5 (PDE-5) inhibitors. PDE-5 inhibitors work by enhancing the effects of nitric oxide, a chemical that relaxes smooth muscle.

Widely used PDE-5 inhibitor compositions are Viagra® (sildenafil citrate) tablets (Pfizer, Inc., NY, N.Y.), Levitra® (vardenafil HCL) tablets (Bayer AG, Leverkusen, Germany), and Clalis® (tadalafil) tablets (Eli Lily and Co., Indianapolis, Ind.). Although these compounds are administered to treat erectile dysfunction by potentiating the vasodilatory effects of nitric oxide on penile blood vessels, systemic vasodilation also occurs. As a result, a headache (due to cerebral vasodilation) is oftentimes induced upon taking PDE-5 inhibitors. The severity of the headache can deter sexual relations or participation in other activities. Given that the headaches are caused by vasodilation, treatment with non-steroidal anti-inflammatory agents, including naproxen, aspirin, and acetaminophen, are generally not effective.

Accordingly, combination therapy with a vasodilator and an active agent that counteracts a side-effect of the vasodilator would be useful. Kits including both a vasodilator and side-effect alleviating agent would also be desirable. In particular, combination therapy that minimizes cerebral vasodilation without compromising the effects of the vasodilator would be desirable.

### SUMMARY

Described here are compositions, methods, and kits for alleviating side-effects of vasodilator therapy. The compositions and kits may include any suitable vasodilator or combination of vasodilators and/or any suitable side-effect alleviating agent or combination of side-effect alleviating agents. They may be designed so that administration of the vasodilator can be combined with administration of a side-effect alleviating agent. The compositions and kits may also be used in treating various medical conditions and various side-effects of vasodilator therapy. For example, when used to treat erectile dysfunction, the compositions and kits may include a PDE-5 inhibitor as the vasodilator and pseudoephedrine as the side-effect alleviating agent to alleviate any associated headache.

In some variations, the vasodilator and side-effect alleviating agent are formulated together into a single composition. The composition may be administered via any suitable route, and may be formulated into any suitable dosage form. Here the composition, or a portion thereof, may be adapted for immediate release, controlled release, delayed release, extended release, or timed release. As further described below, the vasodilator and side-effect alleviating agent may also be formulated in any suitable form that achieves the desired release profile. In some variations, the single composition may be configured to release the side-effect alleviating agent first and then the vasodilator second. In other variations, the single composition may be configured to release the vasodilator first and then the side-effect alleviating agent second. In further variations, the single composition may be configured so that the vasodilator and side-effect alleviating agent are simultaneously released (released at the same time). In some instances the selection of the particular vasodilator or side-effect alleviating agent included in the single composition may depend on their pharmacokinetics, e.g., their metabolism and half life in vivo. The half life of the vasodilator included here, e.g., sildenafil, may match or substantially follow the same pharmacokinetics of the side-effect alleviating agent, e.g., the vasoconstrictor, that is employed. In other instances a PDE-5 inhibitor with a longer half life may be combined with an extended release form of the side-effect alleviating agent (e.g., extended release phenylephrine). In yet other variations, the effect of the vasodilator and side-effect alleviating agent, by virtue of their half life and/or their formulated dosage form in vivo are varied. For example, the effect of the vasodilator may outlast the effect of the side-effect alleviating agent, and vice versa.

When formulated into separate compositions, the vasodilator-containing composition may be administered before the side-effect alleviating composition, or vice versa (sequential administration). The separate compositions may be administered via the same or different routes, or provided in the same or different dosage forms. The dosage forms may also be formulated for immediate release, controlled release, delayed release, extended release, or timed release. Kits may also be made from the compositions described herein, and configured for use with specific medical conditions. For example, the kits may be configured for use with erectile dysfunction treatment.

### DETAILED DESCRIPTION

Described here are compositions for treating side-effects of vasodilator therapy. Methods for administering the compositions, as well as kits with compositions contained therein are also described. The compositions (dosage units) may include both a vasodilator and an active agent that alleviates a side-effect of the vasodilator. Alternatively, the vasodilator and side-effect alleviating agent may each be provided in separate compositions. As used herein, the term “drug” refers to either the vasodilator or the side-effect alleviating agent. Furthermore, as used herein, the terms “alleviate” or “alleviating” refer to reduction (e.g., in severity or reoccurrence), elimination, or prevention of a side-effect. In some instances, a composition may be provided having another agent that neutralizes or negates the effect of the vasodilator. The dosage units may be of any form, e.g., solid, semi-solid, liquid, etc.

The compositions and kits may be used for any medical condition that may benefit from vasodilator therapy. For example, the vasodilator may be used to treat allergic disorders, cardiovascular disorders, gastrointestinal motility disorders, respiratory disorders, and urogenital disorders. In one variation, the medical condition is erectile dysfunction. As used herein, the terms “treat,” “treating,” and “treatment,” refer to the provision of the vasodilator to a patient, or to the resolution, reduction, or prevention of the medical condition, its symptoms, or sequelae.

Furthermore, the side-effect alleviating agent included in the compositions may be used to alleviate any side-effect of vasodilator administration. In some variations, the side-effect alleviating agent may be used to alleviate headaches, including migraine headaches, cluster headaches, tension headaches, and the like. When included in a kit, the compositions may include the side-effect alleviating agent, as well as the vasodilator, in a range of doses. The kits may be designed to target specific medical conditions. The kits may also be packaged such that only compositions having a side-effect alleviating agent is provided, or only a vasodilator is provided.

### I. COMPOSITIONS

The combination therapy described here generally provides a vasodilator for treating a medical condition, and an active agent that alleviates one or more side-effects of the vasodilator. The vasodilator and side-effect alleviating agent may be formulated into a single composition or into separate compositions (i.e., the vasodilator is provided in a composition separate from the composition that provides the side-effect alleviating agent). The compositions may be formulated into any suitable dosage form, including, but not limited to, oral dosage forms, topical dosage forms (e.g., for application to skin or mucosa), inhalable dosage forms, injectable dosage forms, intravenous dosage forms, liposomes, and particulate forms (e.g., microparticles, nanoparticles, etc.). In some variations, the dosage forms, or portions thereof, may be formulated for immediate release, controlled release, delayed release, extended release, or timed release.

Vasodilators

Any suitable vasodilator may be included in the compositions described here. For example, the compositions may include adenosine agonists, alpha blockers, nitrates, or phosphodiesterase inhibitors. In one variation, the compositions include PDE-5 inhibitors. Examples of PDE-5 inhibitors that may be used include, but are not limited to, avanafil, sildenafil, tadalafil, udenafil, vardenafil, horny goat weed, combinations, salts, esters, amides, precursors, analogues, stereoisomers, and derivatives thereof. In some variations, the compositions include sildenafil citrate. Other PDE-5 inhibitors that may be used include pyrazolopyrimidinones, griseolic acid derivatives, 2-phenylpurinone derivatives, phenylpyridone derivatives, fused and condensed pyrimidines, pyrimidopyrimidine derivatives, purine compounds, quinazoline compounds, phenylpyrimidinone derivatives, imidazoquinoxalinone derivatives or aza analogues thereof, and phenylpyridone derivatives.

Selection of the vasodilator to be employed will generally depend on such factors as the medical condition being treated, patient tolerance, interaction with other prescribed medications, and the pharmacokinetics desired. The amount of vasodilator administered and the dosing regimen used, may also depend on the medical condition being treated, but may also depend on the particular vasodilator selected and the age and general health of the individual being treated.

Side-Effect Alleviating Agent

The compositions described herein may include any suitable active agent that alleviates a side-effect of vasodilator therapy. In general, the active agent alleviates the side-effect while maintaining sufficient vasodilation or vasodilation for a sufficient period of time in the target tissue or organ of vasodilator therapy. Given that the side-effects are generally understood to result from vasodilation, the active agents included may have vasoconstrictive properties. In some variations, the active agent employed is a sympathomimetic agent.

Examples of sympathomimetic agents that may be used in the compositions described here include, but are not limited to, adrenergic agonists, methylxanthines, norepinephrine precursors, serotonin precursors, stimulants, triptans, and combinations thereof.

When adrenergic agonists are used, the compositions may include, without limitation, albuterol, adrafinil, adrenalone, amidephrine, apraclonidine, bambuterol, bitolterol, budralazine, carbuterol, clenbuterol, clonidine, clorprenaline, cyclopentamine, denopamine, dimetofrine, dipivefrin, dioxethedrine, dopexamine, ecabapide, etafedrine, fenoterol, formoterol, fenoxazoline, guanabenz, guanfacine, hexoprenaline, ibopamine, indanazoline, isoetharine, isometheptene, isoproternal, isosupine, levalbuterol, mabuterol, mephentermine, metaraminol, metaproterenol, methoxamine, methoxyphenamine, methylhexaneamine, midodrine, mivazerol, modafinil, moxonidine, naphazoline, octodrine, octopamine, oxfedrine, oxymetazoline, pholedrine, pirbuterol, prenalterol, procaterol, propylhexedrine, protokylol, reproterol, rilmenidine, ritodrine, salmeterol, soterenol, talipexole, terbutaline, tetrahydrozoline, tiamenidine, tramazoline, tretoquinol, tuaminoheptane, tulobuterol, tymazoline, tyramine, xamoterol, xylometazoline, combinations, salts, esters, amides, precursors, analogues, and derivatives thereof.

When methylxanthines are used, the compositions may include, without limitation, aminophylline, caffeine, theobromide, and theophylline. L-tyrosine may be used as the norepinephrine precursor, and L-tryptophan as the serotonin precursor.

The triptans that may be used in the compositions described here include, but are not limited to, almotriptan, eletriptan, frovatriptan, naratriptan, rizatriptan, sumatriptan, and zolmitriptan, combinations, salts, esters, amides, precursors, analogues, and derivatives thereof.

In other variations, the compositions may include a stimulant. Examples of stimulants that may be used, include, but are not limited to, amphetamine, benzphetamine, cyclopentamine, dextroamphetamine, diethylpropion, ephedrine, epinephrine, hydroxyamphetamine, methamphetamine, phenylephrine, pseudoephedrine, combinations, salts, esters, amides, precursors, analogues, and derivatives thereof.

Selection of the side-effect alleviating agent to include in the compositions may depend on such factors as the particular vasodilator administered, medical condition of the patient, severity or refractoriness of the side-effect, and the pharmacokinetics desired. For example, a common side-effect of PDE-5 inhibitors used for erectile dysfunction is headache. In view of this, an active agent that counteracts cerebral vasodilation (e.g., a cerebral vasoconstrictor) may be employed in combination with the PDE-5 inhibitor to alleviate the headache induced by the PDE-5 inhibitor.

Dosage Forms

The compositions described here may be formulated into any dosage form, including, but not limited to, oral dosage forms, topical dosage forms, inhalable dosage forms, injectable dosage forms, intravenous dosage forms, and particulate forms. The dosage forms may also be adapted for any type of drug release, e.g., immediate release, controlled release, delayed release, extended release, or timed release. Other ingredients, such as pH buffering agents, binders, disintegrants, diluents, emulsifying agents, fillers, lubricants, penetration enhancers, wetting agents, flavoring agents, colorants, and preservatives, may also be included in the compositions.

Selection of the dosage form to administer may depend on such factors as the particular vasodilator and/or side-effect alleviating agent being delivered, the side-effect being treated, and the type of pharmacokinetics desired. For example, when nausea is the side-effect, it may be desirable to administer the composition as a suppository, sublingual dosage form, or other dosage form in which drug may be delivered without gastrointestinal absorption. When headache is the side-effect, rapid relief may be provided by immediate release dosage forms, dosage forms applied to oral mucosa, inhalable or mist/spray dosage forms, or intravenous dosage forms. A more detailed description of some of these dosage forms is provided below.

1) Oral Dosage Forms

The compositions may be formulated into any suitable oral dosage form. For example, the compositions may be formulated as liquids, tablets, capsules, films, strips, wafers, lonzenges, gums, lollipops, oral mists, etc. The oral dosage forms generally include a vasodilator and/or a side-effect alleviating agent, and are suitable for administration via placement in the mouth, including application to oral mucosal surfaces. In some instances, the oral dosage form, e.g., a tablet, is coated. The coating may include the vasodilator or the side-effect alleviating agent. In other instances, the coating may be used to mask the taste of dosage form ingredients, improve the appearance of the dosage form, enhance surface characteristics, e.g., smoothness, so that they are easier to administer, extend shelf life, or modify release kinetics.

When a coating is employed to modify release kinetics, it may be used to release (deliver) the vasodilator before or after the side-effect alleviating agent, e.g., a vasoconstrictor, the side-effect alleviating agent before the vasodilator, or extend release of the vasodilator or the side-effect alleviating agent so that one is released for a longer period of time than the other.

In some variations, a coating that is insoluble in the gastrointestinal tract may be used. Examples of useful coatings that are substantially insoluble in the gastrointestinal tract include, but are not limited to, coatings comprising a hydrophobic material. In one variation, the coating that is substantially insoluble in the gastrointestinal tract comprises a cellulose polymer. In certain variations, the cellulose polymer is a cellulose ether, a cellulose ester, or a cellulose ester ether. In one variation, the cellulose polymers have a degree of substitution, D.S., on the anhydroglucose unit of from zero up to and including 3. By “degree of substitution” it is meant the average number of hydroxyl groups present on the anhydroglucose-unit of the cellulose polymer that are replaced by a substituting group. Representative cellulose polymers include, but are not limited to, cellulose acylate, cellulose diacylate, cellulose triacylate, cellulose acetate, cellulose diacetate, cellulose triacetate, mono, di, and tricellulose alkanylates, mono, di, and tricellulose aroylates, and mono, di, and tricellulose alkenylates. Exemplary cellulose polymers include cellulose acetate having an acetyl content up to about 21%; cellulose acetate having an acetyl content up to about 32 to 39.8%; cellulose acetate having a D.S. of about 1 to 2 and an acetyl content of about 21 to 35%; and cellulose acetate having a D.S. of about 2 to 3 and an acetyl content of about 35 to 44.8%. In one variation, the cellulose polymer is ethylcellulose, cellulose acetate, cellulose propionate (low, medium, or high molecular weight), cellulose acetate propionate, cellulose acetate butyrate, cellulose acetate phthalate, or cellulose triacetate. In one variation, the ethylcellulose has an ethoxy content of about 44 to 55%.

More specific cellulose polymers may include cellulose propionate having a D.S. of about 1.8 and a propyl content of about 39.2 to 45% and a hydroxyl content of about 2.8 to 5.4%; cellulose acetate butyrate having a D.S. of about 1.8, an acetyl content of about 13 to 15%, and a butyryl content of about 34 to 39%; cellulose acetate butyrate having an acetyl content of about 2 to 29%, a butyryl content of about 17 to 53%, and a hydroxyl content of about 0.5 to 4.7%; cellulose triacylate having a D.S. of about 2.9 to 3 such as cellulose triacetate, cellulose trivalerate, cellulose trilaurate, cellulose tripatmitate, cellulose trisuccinate, and cellulose trioctanoate; cellulose diacylates having a D.S. of about 2.2 to 2.6 such as cellulose disuccinate, cellulose dipalmitate, cellulose dioctanoate, cellulose dipentanoate, and coesters of cellulose such as cellulose acetate butyrate, cellulose acetate octanoate butyrate, and cellulose acetate propionate.

Additional cellulose polymers useful in coatings that are substantially insoluble in the gastrointestinal tract include, but are not limited to, acetaldehyde dimethyl cellulose acetate, cellulose acetate ethylcarbamate, cellulose acetate methylcarbamate, and cellulose acetate dimethylaminocellulose acetate.

Acrylic polymers may also be useful and include, but are not limited to, acrylic resins comprising copolymers synthesized from acrylic and methacrylic acid esters (e.g., the copolymer of acrylic acid lower alkyl ester and methacrylic acid lower alkyl ester) containing about 0.02 to 0.03 moles of a tri (lower alkyl) ammonium group per mole of acrylic and methacrylic monomer. In one variation, the acrylic resin is Eudragit RS 30 D manufactured by Rohm Tech Inc. of Fitchburg, Mass. Eudragit RS 30 D is a water insoluble copolymer of ethyl acrylate (EA), methyl methacrylate (MM) and trimethylammonioethyl methacrylate chloride (TAM) in which the molar ratio of TAM to the remaining components (EA and MM) is 1:40.

Other useful polymers useful for forming an insoluble coating also include, but are not limited to, poly(lactic/glycolic acid) (“PLGA”) copolymers, polylactides, polyglycolides, polyanhydrides, polyorthoesters, polycaprolactones, polyphosphazenes, polysaccharides, proteinaceous polymers, polyesters, polydioxanone, polygluconate, polylactic-acid polyethylene oxide copolymers, poly(hydroxybutyrate), polyphosphoesters, and mixtures thereof.

In other variations, an acid soluble coating may be used. Here the acid soluble coating may be a layer that is substantially soluble at a pH of less than about pH 5.0, but substantially insoluble at a pH of greater than about pH 5.5. In one variation, the acid soluble layer may be substantially soluble at a pH of less than about pH 4.0, but substantially insoluble at a pH of greater than about pH 4.5. In another variation, the acid soluble layer may be substantially soluble at a pH of less than about pH 3.0, but substantially insoluble at a pH of greater than about pH 3.5.

In some variations, the acid soluble coating includes a polymer having a dimethylaminoethyl ammonium functionality. Such a polymer is commercially available as EUDRAGIT E 100 or Eudragit E PO from Rohm Pharma GmbH, Weiterstat, Germany. Examples of other suitable acid soluble polymers can be found in “Materials Used in Pharmaceutical Formulations,” edited by A. T. Florence, Society of Chemical Industries, 1984.

In another variation, a base soluble coating may be used. The base soluble coating may be a layer that is substantially soluble at a pH of greater than about pH 5.5, but substantially insoluble at a pH of less than about 5.0. In one variation, the base soluble layer may be substantially soluble at a pH of greater than about pH 6.5, but substantially insoluble at a pH of less than about 6.0. In another variation, the base soluble layer may be substantially soluble at a pH of greater than about pH 7.5, but substantially insoluble at a pH of less than about 7.0. The base-soluble layer generally comprises a base-soluble polymer. In one variation, the base soluble polymer includes an anionic copolymer of methacrylic acid and methacrylates having carboxylic acid functionalities. Such a polymer is commercially available as EUDRAGIT L 100-55, EUDRAGIT L 30D-55, EUDRAGIT L, or EUDRAGIT S 100 (commercially available from Rohm Pharma GmbH, Weiterstat, Germany). Examples of other suitable base soluble polymers can be found in “Materials Used in Pharmaceutical Formulations,” edited by A. T. Florence, Society of Chemical Industries, 1984. It is understood that any one or combination of insoluble, acid soluble, and base soluble coatings may be included in the solid dosage forms.

For solid oral dosage forms, conventional solid carriers that may be used include, but are not limited to, pharmaceutical grades of mannitol, lactose, starch, magnesium stearate, sodium saccharin, talc, cellulose, glucose, sucrose, magnesium carbonate, and the like. When oral liquids are administered; the liquid compositions may be prepared, for example, by dissolving, dispersing, etc., a vasodilator and/or side-effect alleviating agent in water, saline, aqueous dextrose, glycerol, ethanol, and the like, to thereby form a solution or suspension. Exemplary methods of preparing such dosage forms are known (see, e.g., Remington: The Science and Practice of Pharmacy, 20^{th} Ed. (Baltimore, Md.: Lippincott Williams & Wilkins Publishing, 2000), which is incorporated herein by reference.

In some variations, the oral dosage forms include a penetration enhancer, which may increase the rate at which the vasodilator and/or side-effect alleviating agent passes through mucosal tissue. Examples of suitable penetration enhancers that may be employed include, without limitation, dimethylsulfoxide (DMSO), dimethyl formamide (DMF), N,N-dimethylacetamide (DMA), decylmethylsulfoxide, polyethylene glycol monolaurate, glycerol monolaurate, lecithin, alcohols (e.g., ethanol), and surfactants.

When adhesiveness of the oral dosage forms is desirable, a hydrophilic polymer may be included. Exemplary hydrophilic polymers that may be used include, but are not limited to, acrylic acid polymers, hydrolyzed polyvinylalcohol, polyethylene oxides, polyacrylates, vinyl polymers, polyvinylpyrrolidone, dextran, guar gum, pectins, starches, and cellulosic polymers.

As previously mentioned, other ingredients such as pH buffering agents, disintegrants, diluents, binders, fillers, emulsifying agents, lubricants, penetration enhancers, wetting agents, flavoring agents, colorants, and preservatives may be incorporated into the compositions. For oral dosage forms, examples of buffering agents include, but are not limited to, sodium acetate, sorbitan monolaurate, triethanolamine sodium acetate, or triethanolamine oleate. Exemplary disintegrants that may be used include, but are not limited to, cross-linked polyvinylpyrrolidones (e.g., crospovidone), cross-linked carboxylic methylcelluloses (e.g., croscarmelose), alginic acid, calcium silicate, sodium carboxymethyl starches, methylcellulose, agar bentonite, alginic acid, calcium carbonate, polyoxyethylene sorbitan fatty acid esters, sodium lauryl sulfate, stearic monoglyceride, and lactose.

Suitable diluents to employ in the oral dosage forms are those which are generally useful in pharmaceutical formulations prepared using compression techniques. Exemplary diluents include, but are not limited to, dicalcium phosphate dehydrate, sugars that have been processed by co-crystallization with dextrin, lactose, calcium phosphate, cellulose, kaolin, mannitol, sodium chloride, dry starch, powdered sugar, and the like.

Binders are generally those compounds that enhance dosage form adhesion. Suitable binders that may be used in the oral dosage forms, include, but are not limited to, water, ethanol, polyvinylpyrrolidone, starch, gelatin, or sugars (e.g., sucrose, dextrose, molasses, and lactose). Lubricants that may be used include without limitation, stearic acid, polyethylene glycol, and magnesium stearate. Exemplary wetting agents that may be used are glycerin, starches, and the like.

Conventional flavoring agents may also be incorporated into the dosage forms, such as those described in Remington: The Science and Practice of Pharmacy, 20^{th} Ed. (Baltimore, Md.: Lippincott, Williams and Wilkins Publishing, 2000). When employed, the dosage forms may generally contain from about 0.5% to about 2% by weight of a flavoring agent.

Conventional colorants such as dyes and/or pigments may also be used, such as those described in the Handbook of Pharmaceutical Excipients, by the American Pharmaceutical Association & the Pharmaceutical Society of Great Britain (1986), which is incorporated herein by reference. When included, the dosage forms may generally contain from about 0.5% to about 2% by weight of a colorant.

In some variations, the oral dosage forms, or portions thereof, are made for immediate release, controlled release, delayed release, extended release, or timed release. Dosage forms having one or more of these release rate characteristics may provide the vasodilator or side-effect alleviating agent over a longer period of time, or allow the side-effect alleviating agent to be delivered at a different time then the vasodilator, e.g., before or after the vasodilator. Such a dosage form may comprise a tablet that has an outer layer or coating having a rapidly disintegrating component. For example, a tablet core that includes a PDE-5 inhibitor, e.g., sildenafil, may have a rapidly dissolving outer layer that comprises a vasoconstrictor, e.g., phenylephrine. It should be understood that the PDE-5 inhibitor could be included in the rapidly dissolving outer layer and the vasoconstrictor in the core depending on the type of release profile desired. It should also be understood that the tablet core may be of any suitable form, e.g., solid, semi-solid, liquid, particulate, etc.

In other variations, the oral dosage form may be formulated as a partitioned tablet to that the vasodilator and the side-effect alleviating agent, e.g., a vasoconstrictor, are adjacent one another. For example, the vasodilator composition and vasoconstrictor composition may be compartmentalized, encapsulated, divided, or otherwise separated from each other while being adjacent or next to each other. In another variation, the extended release component may include slowly dissolving particles. In other variations, the dosage form may include an extended release matrix containing rapidly disintegrating particles.

In certain variations, the dosage forms are configured for direct application to the buccal, lingual, or sublingual area to achieve rapid onset. When lingually applied (on the tongue), the dosage forms stimulate saliva production, thus enhancing rapid disintegration of the dosage forms and dissolution of the drug. When applied sublingually, the dosage forms are applied directly to the absorptive membrane on the underside of the tongue. Exemplary dosage forms for use with this type of administration include strips, oral mists, granulated particles, gums, lyophilized wafers/tablets, lozenges, pills, tablets, rapidly disintegrating tablets, troches, and the like.

When granulated particles are used, the particles may have median sizes of about 50 to about 500 microns. In some instances, the median particle size is between about 100 and about 200 microns. The granulated particles may be formed by any of a variety of processes including spheronization, milling, de-agglomeration, precipitation, and/or crystallization.

When in strip or film form, the strip or film will generally be prepared to disintegrate and disperse rapidly and provide for high bioavailability of the drug. The strips may be applied to either or both of the top side or bottom side of the tongue. Strips to be applied under the tongue may be shaped with curved edges in order that the dosage unit may fit comfortably and precisely in the sublingual cavity. In one variation, the dosage form is a rapidly disintegrating tablet, such as a formulation that disintegrates in the mouth within seconds of placement on the tongue, allowing rapid release of the drug. Effervescent agents, such as those described in U.S. Pat. No. 5,178,878, which is incorporated herein by reference, may be included to speed disintegration of the dosage form in the oral cavity.

The oral dosage forms described here may be manufactured using conventional processes. Actual methods of preparing such dosage forms are known. See, e.g., Remington: The Science and Practice of Pharmacy, 20^{th} Ed., (Baltimore, Md.: Lippincott, Williams and Wilkins Publishing, 2000).

2) Topical Dosage Forms

The compositions described herein may be formulated into any topical dosage form. The topical dosage forms may be creams, lotions, solutions, gels, ointments, pastes, patches, etc. The topical dosage forms generally include a vasodilator and/or a side-effect alleviating agent, and are suitable for application to any body surface, including mucosal body surfaces.

Various additives may also be included in the topical dosage forms. For example, solvents, including relatively small amounts of alcohol, may be used to solubilize certain formulation components. Penetration enhancers may also be included. Examples of suitable penetration enhancers include, but are not limited to, ethers such as diethylene glycol monoethyl ether; diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80), and lecithin; alcohols such as ethanol, propanol, octanol, benzyl alcohol, and the like; polyethylene glycol and esters thereof, such as polyethylene glycol monolaurate; amides and other nitrogenous compounds such as urea, dimethylacetamide (DMA), dimethylformamide (DMF), 2-pyrrolidone, 1-methyl-2-pyrrolidone, ethanolamine, diethanolamine, and triethanolamine; terpenes; alkanones; and organic acids; and sulfoxides such as DMSO.

In some variations, the topical dosage form is an ointment. The ointment base may be an oleaginous base, an emulsifiable base, an emulsion base, or a water-soluble base. The oleaginous ointment base that may be used includes, without limitation, vegetable oils, fats obtained from animals, and semisolid hydrocarbons obtained from petroleum. Suitable emulsifiable ointment bases that may be used, include, for example, hydroxystearin sulfate, anhydrous lanolin, and hydrophilic petrolatum. Exemplary emulsion ointment bases that may be used are water-in-oil (W/O) emulsions or oil-in-water (0/W) emulsions that include, for example, cetyl alcohol, glyceryl monostearate, lanolin, and stearic acid.

In other variations, the topical dosage form is a cream. The creams may be viscous liquids or semisolid emulsions, either oil-in-water or water-in-oil. The cream bases may be water-washable, and contain an oil phase, an emulsifier, and an aqueous phase. The oil phase, or internal phase, may be generally comprised of petrolatum and a fatty alcohol such as cetyl or stearyl alcohol. The aqueous phase may be formulated to exceed the oil phase in volume, and contain a humectant.

In, yet another variation, the topical dosage form is a gel. The gels may be semisolid, suspension-type systems. Single-phase gels may contain organic macromolecules distributed substantially uniformly throughout the carrier liquid, which may be aqueous, but may also contain an alcohol and, optionally, an oil. Exemplary organic macromolecules that may be used in the gels, include, but are not limited to, carbomers; hydrophilic polymers such as polyethylene oxides, polyoxyethylene-polyoxypropylene copolymers, and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin.

In yet further variations, the topical dosage form is a lotion. The lotions may be formulated as suspensions of solids and contain suspending agents to produce better dispersions. Examples of such suspending agents include methylcellulose and sodium carboxymethylcellulo se.

The topical dosage forms may also be formulated as a paste. Pastes are semisolid dosage forms in which the active agent is suspended in a suitable base. Depending on the nature of the base, pastes are divided between fatty pastes or those made from a single-phase aqueous gels. The base in a fatty paste is generally petrolatum, hydrophilic petrolatum, or the like. The pastes made from single-phase aqueous gels may generally incorporate carboxymethylcellulose or the like as a base.

In some variations, the topical dosage forms are prepared with liposomes, micelles, or microspheres. Liposomes are microscopic vesicles having a lipid wall comprising a lipid bilayer. Liposome fomiulations may be used for poorly soluble or insoluble drugs. Liposomal preparations for use in the dosage forms described here include cationic (positively charged), anionic (negatively charged), and neutral preparations. Cationic liposomes are readily available. For example, N[1-2,3-dioleyloxy)propyl]-N,N,N-triethylammonium (DOTMA) liposomes are available under the trade name Lipofectin® (GIBCO BRL, Grand Island, N.Y.). Anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), and dioleoylphoshatidyl ethanolamine (DOPE), among others. These materials can also be mixed with DOTMA in appropriate ratios. Methods for making liposomes using these materials are well known.

Micelles are comprised of surfactant molecules arranged so that their polar head groups form an outer spherical shell, while their hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethylammonium chloride, dodecylammonium chloride, polyoxyl 8 dodecyl ether, polyoxyl 12 dodecyl ether, nonoxynol 10, and nonoxynol 30. Micelle formulations for use in the topical dosage forms herein described can be either incorporated into the reservoir of a topical or transdermal delivery system, or into a formulation to be applied to the body surface.

Similarly, microspheres may be incorporated into the topical dosage forms. Like liposomes and micelles, microspheres essentially encapsulate a drug or drug-containing formulation. Microspheres are generally, although not necessarily, formed from synthetic or naturally occurring biocompatible polymers, but may also be comprised of charged lipids such as phospholipids. Preparation of microspheres is well known and described in pertinent texts and literature.

Various additives may also be included in the topical dosage forms. For example, solvents, including relatively small amounts of alcohol, may be used to solubilize certain dosage form components. Penetration enhancers may be added. Examples of suitable penetration enhancers include, but are not limited to, ethers such as diethylene glycol monoethyl ether (available commercially as Transcutol®) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80), and lecithin; alcohols such as ethanol, propanol, octanol, benzyl alcohol, and the like; polyethylene glycol and esters thereof such as polyethylene glycol monolaurate (PEGML); amides and other nitrogenous compounds such as urea, dimethylacetamide (DMA), dimethylformamide (DMF), 2-pyrrolidone, 1-methyl-2-pyrrolidone, ethanolamine, diethanolamine, and triethanolamine; terpenes; alkanones; and organic acids such as citric acid and succinic acid. Azone® and sulfoxides such as DMSO and C₁₀ MSO may also be used.

The topical dosage forms may also include conventional additives such as opacifiers, antioxidants, fragrance, colorants, gelling agents, thickening agents, stabilizers, surfactants, and the like. Other agents may also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof.

The dosage forms may also contain irritation-mitigating additives to minimize or eliminate the possibility of skin irritation resulting from the vasodilator, side-effect alleviating agent, or other components of the composition. Suitable irritation-mitigating additives include, for example, alpha.-tocophetol; monoamine oxidase inhibitors, e.g., phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine.

In other instances, it may be desirable to administer the vasodilator and/or the side-effect alleviating agent using a skin patch. The patch generally includes a drug, e.g., a vasodilator or side-effect alleviating agent, in a layer, or “reservoir,” underlying an upper backing layer. The laminated structure may contain a single reservoir, or it may contain multiple reservoirs. When multiple reservoirs are employed, they may include the same drug or different drugs, or each reservoir may include a combination of drugs. The patches may also be configured to include a component that modifies delivery of a drug therefrom. The patch may be configured to release the vasodilator, e.g., sildenafil, prior to release of the side-effect alleviating agent, e.g., a vasoconstrictor, and vice versa. In some instances, the release of one drug may overlap all or a portion of the release of the other drug. In other instances, the release of one drug may extend beyond the release of the other from the patch. For example, a rate-limiting membrane may be placed between the reservoirs to modify release of the drug.

In some variations, the reservoirs may comprise a polymeric matrix of a pharmaceutically acceptable adhesive material that serves to affix the patch to the skin. For example, the adhesive material may be a pressure-sensitive adhesive (PSA) including, but not limited to, polyethylenes; polysiloxanes; polyisobutylenes; polyacrylates; polyacrylamides; polyurethanes; plasticized ethylene-vinyl acetate copolymers; and tacky rubbers such as polyisobutene, polybutadiene, polystyrene-isoprene copolymers, polystyrene-butadiene copolymers, and neoprene (polychloroprene).

The backing layer functions as the primary structural element of the patch and provides the device with flexibility and in certain variations, occlusivity. The material used for the backing layer will generally be inert and incapable of absorbing the vasodilator, side-effect alleviating agent contained within the reservoirs of the patch. The backing may be comprised of a flexible elastomeric material that serves as a protective covering to prevent loss of drug and/or carrier via transmission through the upper surface of the patch, and may impart a degree of occlusivity to the patch, such that the area of the body surface covered by the patch becomes hydrated during use. The material used for the backing layer may permit the patch to follow the contours of the skin and be worn comfortably on areas of skin such as at joints or other points of flexure that are normally subjected to mechanical strain, with little or no likelihood of the patch disengaging from the skin due to differences in the flexibility or resiliency of the skin and the patch. The materials used as the backing layer may be either occlusive or permeable, as noted above, and may be made from synthetic polymers (e.g., polyester, polyethylene, polypropylene, polyurethane, polyvinyl chloride, and polyether amide), natural polymers (e.g., cellulosic materials), or macroporous woven and nonwoven materials.

During storage and prior to use, the laminated structure may include a release liner. Immediately prior to use, this layer is typically removed from the device so that the patch may be affixed to the skin. The release liner may be made from a drug/carrier impermeable material, and may be prepared as a disposable element that serves only to protect the patch prior to application. The release liner may be formed from a material impermeable to the vasodilator and side-effect alleviating agent, and which is easily stripped from the patch prior to use.

In another variation, the drug-containing reservoir and skin contact adhesive are present as separate and distinct layers, with the adhesive underlying the reservoir. In such a case, the reservoir may be a polymeric matrix as described above. Alternatively, the reservoir may be comprised of a liquid or semisolid formulation contained in a closed compartment or “pouch,” or it may be a hydrogel reservoir, or it may take some other form. Hydrogels are generally macromolecular networks that absorb water and thus swell, but may or may not dissolve in water. That is, hydrogels contain hydrophilic functional groups that provide for water absorption, but the hydrogels are comprised of crosslinked polymers that may give rise to aqueous insolubility. Generally, then, hydrogels are comprised of crosslinked hydrophilic polymers such as a polyurethane, a polyvinyl alcohol, a polyacrylic acid, a polyoxyethylene, a polyvinylpyrrolidone, a poly(hydroxyethyl methacrylate) (poly(HEMA)), or a copolymer or mixture thereof.

Additional layers, e.g., intermediate fabric layers and/or rate-controlling membranes, may also be present in any of the patches. Fabric layers may be used to facilitate fabrication of the patch, while a rate-controlling membrane may be used to control the rate at which a component permeates out of the patch. The component may be a vasodilator, side-effect alleviating agent, a penetration enhancer, or some other component contained in the patch. A rate-controlling membrane, if present, will be included in the patch on the skin side of one or more of the drug reservoirs. The materials used to form such a membrane may be selected to limit the flux of one or more components contained in the patch. Representative materials useful for forming rate-controlling membranes include polyolefins such as polyethylene and polypropylene, polyamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, and the like.

The patches may be fabricated using conventional coating and laminating techniques known in the art. For example, adhesive matrix systems can be prepared by casting a fluid admixture of adhesive, active agent, and carrier onto the backing layer, followed by lamination of the release liner. Similarly, the adhesive mixture may be cast onto the release liner, followed by lamination of the backing layer. Alternatively, the drug reservoir may be prepared in the absence of drug or excipient, and then loaded by “soaking” in a drug/carrier mixture. In general, these patches are fabricated by solvent evaporation, film casting, melt extrusion, thin film lamination, die cutting, or the like.

In certain variations, an adhesive overlayer that also serves as a backing for the patch is used to better secure the patch to the body surface. This overlayer is sized such that it extends beyond the drug reservoir so that adhesive on the overlayer comes into contact with the body surface. The overlayer is useful because the adhesive/drug reservoir layer may lose its adhesion a few hours after application due to hydration. By incorporating such an adhesive overlayer, the patch remains in place for the required period of time.

3) Other Dosage Forms

The vasodilator and side-effect alleviating agent may also be formulated into other parental dosage forms. The drugs may be formulated for administration by injection, e.g., by bolus injection or continuous infusion. Such dosage forms may be prepared by dissolving, suspending, or emulsifying the drugs in an aqueous or nonaqueous solvent, such as vegetable or other similar oils, synthetic aliphatic acid glycerides, esters of higher aliphatic acids or propylene glycol, and if desired, with conventional additives such as solubilizers, isotonic agents, suspending agents, emulsifying agents, stabilizers and preservatives.

In one variation, the injectable dosage form is prepared as an aqueous solution, using Hanks's solution, Ringer's solution, or normal saline. Formulations for injection may be presented in unit dose form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

The injectable dosage form may further be prepared as an oily suspension of drug. Suitable lipophilic solvents or vehicles for use in this instance include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the drugs to allow for the preparation of highly concentrated solutions. Alternatively, the drugs may be in powder form for constitution with a suitable vehicle, e.g., sterile water, normal saline, etc., before use.

In other variations, the vasodilator and/or the side-effect alleviating agent are formulated to be delivered as a mist or aerosol. The mist or aerosol may be administered lingually, buccally, or sublingually, or may be inhaled so that the mist or aerosol particles flow into the respiratory passages. The aerosol may be delivered via a dry powder inhaler, metered-dose inhaler, breath-actuated inhaler, and the like, or may be delivered from a pressurized container, non-pressurized dispenser, pump, or nebulizer with the use of a suitable propellant. Propellants include, but are not limited to, dichlorodifluoromethane, trichlorofluoromethane, dichlorotetrafluoroethane, hydrofluoroalkanes, carbon dioxide, or inert gases. Hydrofluoroalkanes include 1,1,1,2-tetrafluoroethane or 1,1,1,2,3,3,3-heptafluoropropane. Inert gasses include nitrogen or argon. In the case of a pressurized aerosol, the dosage unit may be determined by providing a valve to deliver a metered amount. The pressurized container, pump, spray, or nebulizer may contain a solution or suspension of the drug, e.g., using a mixture of ethanol and the propellant as the solvent, which may additionally contain a lubricant, e.g., sorbitan trioleate.

The vasodilator and/or side-effect alleviating agent may also be formulated as a conjugate that is capable of releasing one or both of the vasodilator and the side-effect alleviating agent. For example, after administration of a conjugate comprising a vasodilator, e.g., sildenafil, and a side-effect alleviating agent, e.g., a vasoconstrictor such as phenylephrine, the conjugate may be configured to release the vasodilator and/or side-effect alleviating agent in a predetermined manner. For example, the conjugate may be configured to release the vasodilator first and the side-effect alleviating agent second, and vice versa. The conjugates may also be configured to release the drugs in any suitable manner, e.g., immediate release, controlled release, delayed release, and timed release fashions, and the like. In some variations, the conjugate joins the vasodilator and side-effect alleviating agent via a linker molecule. The structure of the linker molecule will generally vary depending on the particular vasodilator and side-effect alleviating agent employed. In some variations, the linker may be an oligosaccharide, e.g., a cyclodextrin. In other variations, the linker may be a polymer capable of binding to one or both drugs via a nucleophilic group, e.g., amines, thiols, hydroxyls, hydroxylamines, hydrazines, amides, guanadines, imines, aromatic rings, and nucleophilic carbon atoms. In yet further variations, polyethylene glycols may be attached (PEGylation) to one or both of the vasodilator and side-effect alleviating agent in order to conjugate them to one another.

### II. METHODS

### 1) Administration

The compositions described here may be administered in any suitable manner. For example, the compositions may be administered via oral, topical (including transdermal and transmucosal routes) intravenous, subcutaneous, intramuscular, and rectal routes. The compositions may also be administered by inhalation using spray devices or devices such as nebulizers, metered-dose inhalers, breath-actuated inhalers, and dry powder inhalers. The compositions may also be administered so that the benefit (effect) of the vasodilator and side-effect alleviating agent overlaps for some period of time, occurs at the same time, or occurs at separate times.

As previously mentioned, the vasodilator and active agent that alleviates a side-effect of the vasodilator are administered in combination with one another. In some variations, combined administration occurs as a result of having the vasodilator and side-effect alleviating agent in a single dosage form. In other variations, e.g., when the vasodilator and side-effect alleviating agent are provided separately, each in its own dosage form, combined administration results from administering the vasodilator and side-effect alleviating agent concurrently (i.e., simultaneously) or sequentially (e.g., after a certain time period has elapsed from administration of the first dosage form, or after a side-effect has developed). For example, a PDE-5 inhibitor such as sildenafil citrate and a drug that alleviates headaches may be administered concurrently or sequentially (within seconds, minutes, hours, or days). Administration of the side-effect alleviating agent and vasodilator may be repeated as often as desired.

### 2) Dosing Regimens

The dosing regimen employed may depend on a number of factors, such as the severity of the underlying medical condition, responsiveness of the medical condition to vasodilator therapy, the particular side-effect being alleviated, and the severity and risk of recurrence of the side-effect. The dosing regimen may provide one or more doses per day of the vasodilator and/or the side-effect alleviating agent, and may continue for several hours, for one day to several days, or for several months or more. In general, the dosing regimen will continue until the underlying medical condition is treated or until the side-effect is alleviated.

The vasodilator and side-effect alleviating agent may be provided in any dose. When a PDE-5 inhibitor is used, a typical daily dose of PDE-5 inhibitor to be administered may be from about 0.5 mg to about 100 mg, from about 0.5 mg to about 50 mg, from about 0.5 mg to about 25 mg, from about 0.5 mg to about 10 mg, or from about 0.5 mg to about 3 mg. Depending on the half-life of the PDE-5 inhibitor and the availability via the chosen route of administration, the dosing regimen may be modulated in order to achieve satisfactory therapeutic results. Dosage forms requiring transmucosal or gastrointestinal absorption may include higher doses of the PDE-5 inhibitor.

The compositions may include the vasodilator or the side-effect alleviating agent in any amount. For example, they may be included in amounts of about 1% to about 99% by weight of the composition. In one variation, the vasodilator is included in an amount of about 1% to about 30% by weight of the composition. In other variations, the vasodilator and side-effect alleviating agent are included in the compositions according to a specific ratio. For example, the vasodilator and side-effect alleviating agent, respectively, may be included in a ratio of about 1:1, about 1:1.5, about 1:2, about 1:2.5, about 1:3, about 1:3.5, or about 1:4, by weight. It is understood that the above dosages are exemplary, and that there may be instances in which higher or lower dosages may be merited.

### 3) Medical Conditions Treated and Side-Effects Alleviated

The compositions described herein may be used to treat any medical condition that may benefit from vasodilator therapy. For example, the vasodilator included in the compositions may be used to treat medical conditions such as allergic disorders, cardiovascular disorders, endocrine disorders, gastrointestinal motility disorders, mood disorders, respiratory disorders, and urogenital disorders. These disorders include, without limitation, erectile dysfunction, portal hypertension, angina, stroke, anal fissures, nutcracker esophagus, hypoxic vasoconstriction, Raynaud's disease, scleroderma, diffuse cutaneous systemic sclerosis, congestive heart failure, ischemic heart disease, pulmonary hypertension, acute respiratory distress syndrome, benign prostatic hypertrophy, autoimmune diseases, overactive bladder, bladder outlet obstruction, incontinence, cancer, diabetes, dysmenorrhea, elevated intra-ocular pressure, glaucoma, glomerular renal insufficiency, hyperglycemia, hypertension, impaired glucose tolerance, inflammatory diseases, insulin resistance syndrome, macular degeneration, nephritis, optic neuropathy, osteoporosis, peripheral arterial disease, polycystic ovarian syndrome, renal failure, thrombocytopenia, and tubular interstitial diseases.

In some variations, the compositions are used with medical disorders in which PDE-5 inhibitor therapy is indicated, or considered to be beneficial. The biochemical, physiological, and clinical effects of PDE-5 inhibitors suggest their utility in a variety of conditions in which modulation of smooth muscle, renal, hemostatic, inflammatory, and/or endocrine function is desirable. Conditions treated by PDE-5 inhibitors include, but are not limited to, erectile dysfunction, premature ejaculation, female sexual dysfunction, cardiovascular, cerebral stroke, congestive heart failure, cerebrovascular conditions, ischemic heart disease, pulmonary arterial hypertension, acute respiratory distress syndrome, benign prostatic hypertrophy, angina, autoimmune diseases, overactive bladder, bladder outlet obstruction, incontinence, cachexia, cancer, diabetes, endarterectomy, diseases characterized by disorders of gut motility, dysmenorrhea, elevated intra-ocular pressure, glaucoma, glomerular renal insufficiency, hyperglycemia, hypertension, impaired glucose tolerance, inflammatory diseases, insulin resistance syndrome, macular degeneration, nephritis, optic neuropathy, osteoporosis, peripheral arterial disease, polycystic ovarian syndrome, renal failure, respiratory tract disorders, thrombocytopenia, tubular interstitial diseases, and urogenital disorders.

Exemplary allergic disorders include, but are not limited to, urticaria, eczema, and rhinitis.

Exemplary cardiovascular disorders include, but are not limited to, hypertension, coronary artery disease, angina pectoris, arrhythmia, cardiovascular diseases associated with hormone replacement therap, cerebral infarction, cerebral ischemia, conditions of reduced blood vessel patency (e.g., postpercutaneous transluminal coronary or carotid angioplasty, or post-bypass surgery graft stenosis), deep vein thrombosis, disseminated intravascular coagulation syndrome, heart failure, migraine, myocardial infarction, peripheral vascular disease, Raynaud's disease, renal ischemia, stroke, venous thromboembolism, pulmonary arterial hypertension, congestive heart failure, and myocardial infarction.

Disorder affecting of gut motility include, but are not limited to, irritable bowel syndrome, diabetic gastroparesis and dyspepsia.

Respiratory tract disorders may include, but are not limited to, acute respiratory failure, allergic asthma, allergic rhinitis, bronchitis, chronic asthma, and reversible airway obstruction.

Other medical conditions for which a PDE-5 inhibitor is indicated, and for which treatment with the compositions described here may be useful include, but are not limited to, pre-eclampsia, Kawasaki disease, multiple sclerosis, diabetic nephropathy, Alzheimer's disease, and psoriasis.

In some variations, the vasodilator is used to treat erectile dysfunction. The erectile dysfunction may be secondary to another medical condition or a side-effect of a prescribed medication. For example, the underlying cause of the erectile dysfunction may be a neurogenic disorder, an endocrine disorder or hormonal imbalance, a cardiovascular disorder, Peyronie's disease, a mood disorder, or a complication of surgery or radiation therapy.

Specifically, erectile dysfunction may be secondary to vasculogenic factors, such as alterations in blood flow to and from the penis. This is thought to be the most frequent organic cause of erectile dysfunction. Common risk factors for vasculogenic erectile dysfunction include hypertension, diabetes, cigarette smoking, and pelvic trauma. Neurogenic erectile dysfunction is generally associated with spinal cord injury, multiple sclerosis, peripheral neuropathy caused by diabetes or alcoholism, or injury to nerves near the penis as a consequence of prostate surgery. Erectile dysfunction is also associated with disturbances in endocrine function resulting in low circulating testosterone levels and elevated prolactin levels.

The side-effect alleviating agent included in the compositions described herein may be used to treat any side-effect of vasodilator therapy. For example, the side-effect may be abnormal vision, chest pain, diarrhea, dizziness, dyspepsia, fluid retention, flushing, nasal congestion, nausea, palpitations, rapid heartbeat, or vomiting. In some variations, the side-effect is a headache. Headaches include migraine headaches, cluster headaches, and tension headaches.

### III. KITS

The compositions may be provided in a kit and include any vasodilator, any side-effect alleviating agent, or any combination thereof. In general, the kits will include one or more vasodilators, one or more active agents that alleviate a side-effect of the vasodilator, and instructions for use. The included compositions may be of the same dosage form or different dosage forms. The kits may also provide each composition as separately packaged units. Instructions may be in written or pictograph form, or can be on recorded media including audio tape, audio CD, video tape, DVD, CD-ROM, or the like.

In some variations, the vasodilator included in the kit may be provided with the side-effect alleviating agent in a single dosage form. In other variations, the vasodilator may be included in the kit in a dosage form separate from dosage form including the side-effect alleviating agent. The kits may also be formed to only include side-effect alleviating agent compositions. The compositions may contain the vasodilator and side-effect alleviating agent in any dose. In some instances, a range of doses is provided.

The kits may be designed to target specific medical conditions. In one variation, the kit is designed for use with erectile dysfunction treatment. Such a kit may include one or more PDE-5 inhibitor compositions and one or more compositions for alleviating headaches. For example, the erectile dysfunction kit may provide a sildenafil citrate tablet(s), and a composition(s) including pseudoephedrine.

### IV. EXAMPLES

### Example 1

### Combination of Viagra and Sudafed

A 65 year-old patient with erectile dysfunction secondary to low testosterone complained of inability to use Viagra because of headaches. Previous administration of caffeine prior to taking the Viagra did not help. After taking one Sudafed (pseudoephedrine hydrochloride) tablet with only minimal relief, the patient took two Sudafed tablets concomitantly with the Viagra. He later reported that the headache was still present but had abated enough to where he could enjoy the experience.

### Example 2

### Combination of Viagra and Sudafed

A patient in his fifties without erectile dysfunction who, although bothered by headaches from Viagra, was still able to have sex. After taking one Sudafed tablet with the Viagra, he did not develop a headache.

### Example 3

### Combination of Viagra and Triptan

A patient in his twenties with a history of migraines had difficulty sustaining an erection for long periods of time. He experienced intolerable headaches with Viagra. Ibuprofen and acetaminophen had no effect on the headache. He reported that administration of a 5 mg tablet of rizatriptan with the Viagra made the headache tolerable. The Viagra also seemed to prolong his erection.

### Example 4

### Combination of Clalis, Ergotamine, and Caffeine

A patient in his forties presented with erectile dysfunction secondary to medications and hemicrania continua (a largely refractory condition consisting of a constant one-sided headache). Viagra and Clalis exacerbated his headaches. As a result, successively higher doses of a triptan were prescribed, but failed to alleviate the headaches. He was then prescribed cafergot, a combination of ergotamine and caffeine, at a dose of 1 mg and instructed to take this with the Clalis. This combination did not worsen his headaches. He was barely able to maintain an erection, but this was an improvement over all other recent attempts at having sexual relations.

### Example 5

### Combination of Viagra and Imitrex

A patient in his late twenties experienced intermittent erectile dysfunction due to stress. He took Viagra but could not tolerate the headache it gave him. Sudafed and caffeine did not relieve the headache A trial of Imitrex (sumatriptan succinate) nasal spray at a dose of 25 mg alleviated the headache to the point where he could enjoy the sex.

### Example 6

### Combination of Viagra and L-Tryptophan

A patient in his fifties with psychologic, rather than physiologic, erectile dysfunction was experiencing headaches after taking Viagra. After trying L-tryptophan supplements the night before sexual intercourse, he reported headache-free sex.

### Example 7

### Combination of Viagra and Albuterol

A patient in his late forties who suffered from headaches with Viagra tried taking the Viagra with albuterol. Specifically, he took two puffs of his albuterol inhaler after developing a headache from the Viagra. He reported that his headache was alleviated by the albuterol.

1. A composition for treating side-effects of vasodilation comprising a vasodilator and an active agent that alleviates a side-effect of the vasodilator.

2. The composition of claim 1 wherein the side-effect is selected from the group consisting of abnormal vision, chest pain, diarrhea, dizziness, dyspepsia, fluid retention, flushing, headache, nasal congestion, nausea, palpitations, rapid heartbeat, and vomiting.

3. The composition of claim 1 wherein the vasodilator comprises an adenosine agonist, an alpha blocker, a nitrate, a PDE-5 inhibitor, or combinations thereof.

4. The composition of claim 3 wherein the vasodilator comprises a PDE-5 inhibitor.

5. The composition of claim 4 wherein the PDE-5 inhibitor comprises avanafil, sildenafil, tadalafil, udenafil, vardenafil, horny goat weed, combinations, salts, or derivatives thereof.

6. The composition of claim 5 wherein the PDE-5 inhibitor comprises sildenafil.

7. The composition of claim 1 wherein the active agent comprises a sympathomimetic agent.

8. The composition of claim 7 wherein the sympathomimetic agent is selected from the group consisting of adrenergic agonists, methylxanthines, norepinephrine precursors, serotonin precursors, stimulants, triptans, and combinations thereof.

9. The composition of claim 8 wherein the active agent comprises an adrenergic agonist.

10. The composition of claim 9 wherein the adrenergic agonist is selected from the group consisting of albuterol, formoterol, isoproternal sulfate, isosupine hydrochloride, levalbuterol, and salmeterol.

11. The composition of claim 8 wherein the active agent comprises a methylxanthine.

12. The composition of claim 11 wherein the methylxanthine is selected from the group consisting of aminophylline, caffeine, theobromide, and theophylline.

13. The composition of claim 8 wherein the active agent comprises a norepinephrine precursor.

14. The composition of claim 13 wherein the norepinephrine precursor comprises L-tyrosine.

15. The composition of claim 8 wherein the active agent comprises a serotonin precursor.

16. The composition of claim 14 wherein the serotonin precursor comprises L-tyrptophan or L5-hydroxytryptophan.

17. The composition of claim 8 wherein the triptan is selected from the group consisting of almotriptan, eletriptan, frovatriptan, naratriptan, rizatriptan, sumatriptan, and zolmitriptan.

18. The composition of claim 8 wherein the active agent comprises a stimulant.

19. The composition of claim 18 wherein the stimulant is selected from the group consisting of amphetamine, amphetamine sulfate, benzphetamine hydrochloride, cyclopentamine hydrochloride, dextroamphetamine hydrochloride, diethylpropion hydrochloride, ephedrine, ephedrine hydrochloride, epinephrine, epinephrine bitartrate, hydroxyamphetamine hydrobromide, methamphetamine hydrochloride, phenylephrine hydrochloride, pseudoephedrine, and combinations thereof.

20. The composition of claim 1 wherein the vasodilator and the active agent are formulated as an oral dosage form, a topical dosage form, an injectable dosage form, an intravenous dosage form, or particles.

21. The composition of claim 20 wherein the vasodilator and the active agent are formulated as an oral dosage form.

22. The composition of claim 21 wherein the oral dosage form is selected from the group consisting of tablets, capsules, films, strips, wafers, lozenges, gums, liquids, rapidly dissolving dosage forms, liposomes, microparticles, and nanoparticles.

23. The composition of claim 21 wherein the oral dosage form further comprises a coating.

24. The composition of claim 20 wherein the topical dosage form is selected from the group consisting of creams, lotions, solutions, gels, ointments, pastes, and patches.

25. The composition of claim 20 wherein one or more of the vasodilator and active agent is formulated for immediate release.

26. The composition of claim 20 wherein one or more of the vasodilator and active agent is formulated for extended release.

27. A method for treating side-effects of vasodilation comprising administering a vasodilator in combination with an active agent that alleviates a side-effect of the vasodilator.

28. The method of claim 27 wherein the vasodilator and active agent that alleviates a side-effect of the vasodilator are administered by oral, topical, intravenous, subcutaneous, intramuscular, rectal, or inhalation routes.

29. The method of claim 27 wherein the vasodilator and the active agent are administered in a single dosage form.

30. The method of claim 27 wherein the vasodilator and the active agent are administered as separate dosage forms.

31. The method of claim 30 wherein the separate dosage forms are different dosage forms.

32. The method of claim 27 wherein the vasodilator and the active agent are administered concurrently.

33. The method of claim 27 wherein the vasodilator and the active agent are administered sequentially.

34. The method of claim 27 wherein the vasodilator and the active agent are joined by a linker molecule.

35. The method of claim 34 wherein the linker molecule releases the vasodilator before release of the active agent.

36. The method of claim 34 wherein the linker molecule releases the active agent before the vasodilator.

37. The method of claim 27 further comprising repeating administration of the active agent that alleviates a side-effect of the vasodilator.

38. The method of claim 27 wherein the vasodilator is administered to treat a medical condition selected from the group consisting of erectile dysfunction, pulmonary hypertension, portal hypertension, angina, stroke, anal fissures, nutcracker esophagus, hypoxic vasoconstriction, Raynaud's disease, scleroderma, diffuse cutaneous systemic sclerosis, mood disorders, and urogential disorders.

39. The method of claim 27 wherein the vasodilator is administered to treat erectile dysfunction.

40. The method of claim 27 wherein the active agent is administered to alleviate headache as the side-effect of the vasodilator.

41. A kit for treating side-effects of vasodilation comprising:
a) a vasodilator;
b) an active agent that alleviates a side-effect of the vasodilator; and
c) instructions for use.

42. The kit of claim 41 wherein the vasodilator and the active agent are included in a single dosage form.

43. The kit of claim 41 wherein the kit comprises separate dosage forms of the vasodilator and the active agent.

44. The kit of claim 43 wherein the separate dosage forms are the same dosage form.

45. The kit of claim 43 wherein the separate dosage forms are different dosage forms.

46. The kit of claim 41 wherein kit comprises different doses of the vasodilator.

47. The kit of claim 41 wherein the kit comprises different doses of the active agent.

48. The kit of claim 41 wherein the vasodilator comprises a PDE-5 inhibitor.
